(19) United States
(12) Patent Application Publication
(10) Pub. No.: US 2012/0016348 A1
(43) Pub. Date: Jan. 19, 2012

(54) ANGIOCATHETER WITH IMPROVED FLOW TECHNIQUE

(76) Inventor: Darrin Hansen, Sandy, UT (US)

(21) Appl. No.: 12/837,731

(22) Filed: Jul. 16, 2010

(51) Int. Cl. A61M 27/00

(52) U.S. Cl. 604/541

(57) ABSTRACT

A catheter for draining of fluid comprising: a body portion; a rim, where said body portion extends from rim; a plurality of holes extending along the length of the body; and a tab, where said tab extends from the rim. The rim includes an opening for the insertion of a needle. The catheter may be in a range of 16 gauge to 18 gauge and in one exemplary embodiment the catheter is three inches in length.

### BACKGROUND OF THE INVENTION

1. Field of Invention

The present invention relates to a catheter that includes a plurality of openings along the length thereof to provide for improved flow of fluids through the catheter.

2. Description of Related Art

An intravascular catheter is a small diameter tube inserted into an individual's to allow for the withdrawal or addition of fluid. Catheters are primarily inserted into blood vessels through the bore of an insertion needle or over the outside of an insertion needle. The catheter provides a means for the insertion of fluids such as medicine or the withdrawal of fluids such as blood. Catheters are also inserted into other tissues to withdraw either cells or fluid typically for testing purposes. Many times the catheter is inserted into a person who may have excessive fluid buildup either after surgery or due to infection that needs to be drained. The common procedure for draining this fluid is the insertion of an angio-catheter and the withdrawal or drainage of the fluid through the catheter. Many times the catheters have a single hole for the transfer of fluid therethrough. With a single hole, tissue, blood or other solid material may obstruct the passage and therefore hinder effective drainage flow through the catheter. Further fluid backup can occur due to the use of a single hole in the catheter. Consequently, a single hole catheter can be difficult when performing a draining function in aspirating a particular tissue.

It would therefore be advantageous to have an angio-catheter that includes perforated tubing to prevent fluid backup and leakage. Such a device could provide a plurality of holes for the withdrawal of fluid through the catheter and provide a more effective means than the single-holed catheter of the prior art.

### SUMMARY OF THE INVENTION

The present invention relates to a catheter for draining of fluid comprising: a body portion; a rim, where said body portion extends from rim; a plurality of holes extending along the length of the body; and a tab, where said tab extends from the rim. The rim includes an opening for the insertion of a needle. The catheter may be in a range of 16 gauge to 18 gauge and in one exemplary embodiment the catheter is three inches in length.

### BRIEF DESCRIPTION OF DRAWINGS

FIG. 1 depicts a catheter with multiple fluid openings according to the present invention.

### DETAILED DESCRIPTION

The present invention provides a catheter that may be used in an office setting or immediately after surgery to remove fluids that have built up in human tissue. The catheter according to the present invention includes a plurality of side holes along the length of the catheter that provided multiple openings for the flow of fluid therethrough. This use of multiple fluid holes therefore decreases the likelihood of any clogging while the draining of fluid is taking place. The catheter according to the present invention is very effective in removing fluid from tissue in an effective manner.

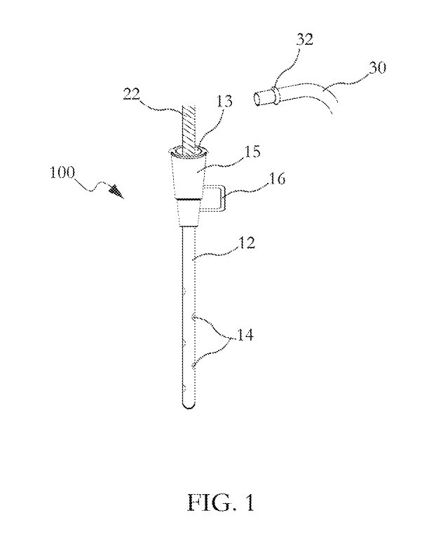

A Catheter 100 according to the present invention is depicted in FIG. 1. The Catheter 100 depicted in the detailed view shows the catheter Body 12 extending downward to the distal end that includes a plurality of Holes 14 along the length of the Body 12. The Holes 14 extend along the length of the Body 12 and provide an additional means for the removal of fluid from tissue. The Catheter 100 may be inserted by use of a Needle 22 which is shown to be inserted into an Opening 13 at the top of the Catheter 100. The top Rim 15 provides a means to support and secure the Catheter 100 during use thereof.

Extending out from one side of the top Rim 15 is a Tab 16 that may be used to anchor the catheter to the skin if the catheter needs to be left in place for an extended period of time. Along the Body 12, the plurality of Holes 14 may be increased or decreased in number or size as desired for various designs of the catheter according to the present invention. Although five holes are depicted along the Body 12 more holes may be provided or even less holes depending on the needs of the medical personnel. In one particular embodiment, the catheter is about 16 to 18-gauge and three inches in length. The plurality of holes along the body of the catheter again may be varied depending on the length and gauge of the catheter. The catheter may be manufactured of a plastic material and suitable for use both a surgical setting or in a physician's office. Also depicted in FIG. 1 is a Tug 30 that may be inserted into the catheter to transport the fluid via a suction device. The Tug 30 includes a Rim 32 that provides the means to secure the Tug 30 into the Opening 13 of the catheter. The Tug 30 may be used in conjunction with the Tab 16, to hold the catheter stationary. The instant invention has been shown and described in what it considers to be the most practical and preferred embodiments. It is recognized, however, that departures may be made there from within the scope of the invention and that obvious modifications will occur to a person skilled in the art.

What is claimed is:

1. A catheter for draining of fluid comprising:
a. a body portion;
b. a rim, where said body portion extends from rim;
c. a plurality of holes extending along the length of the body; and
d. a tab, where said tab extends from the rim.

2. The catheter according to claim 1, where said rim includes an opening for the insertion of a needle.

3. The catheter according to claim 1, where said catheter is in a range of 16 gauge to 18 gauge.

4. The catheter according to claim 1, where said catheter is three inches in length.

5. A catheter for draining of fluid comprising:
a. a body portion;
b. a rim, where said body portion extends from rim;
c. at least two holes extending along the length of the body; and
d. a tab, where said tab extends from the rim.

6. The catheter according to claim 1, where said rim includes an opening for the insertion of a needle.

7. The catheter according to claim 1, where said catheter is in a range of 16 gauge to 18 gauge.

8. The catheter according to claim 1, where said catheter is three inches in length.
